(19) United States
(12) Patent Application Publication
(10) Pub. No.: US 2023/0352158 A1
(43) Pub. Date: Nov. 2, 2023

(54) INFORMATION PROCESSING APPARATUS, SYSTEM, AND METHOD

(71) Applicant: TOYOTA JIDOSHA KABUSHIKI KAISHA, Toyota-shi (JP)

(72) Inventors: Yoshikazu JIKUHARA, Miyoshi-shi (JP); Seii SAI, Yokohama-shi (JP); Ibuki SHIMADA, Miyoshi-shi (JP); Takahiro AOKI, Saitama-shi (JP); Keishi KINOSHITA, Setagaya-ku (JP)

(21) Appl. No.: 18/309,859

(22) Filed: May 1, 2023

(30) Foreign Application Priority Data
May 2, 2022 (JP) 2022-076325

(51) Int. Cl. G16H 40/20; G16H 40/67

(52) U.S. Cl. CPC G16H 40/20; G16H 40/67

(57) ABSTRACT

An information processing apparatus includes a controller. In a case in which a user whose physical condition has worsened or who is suspected of being ill is detected during a medical checkup, the controller determines a hospital to which the user is to be transported from among a plurality of hospitals located within a predetermined range from a site of the medical checkup.

### CROSS-REFERENCE TO RELATED APPLICATION

This application claims priority to Japanese Patent Application No. 2022-076325 filed on May 2, 2022, the entire contents of which are incorporated herein by reference.

### TECHNICAL FIELD

The present disclosure relates to an information processing apparatus, a system, and a method.

### BACKGROUND

Technology for conducting medical checkups in vehicles is known. For example, Patent Literature (PTL) 1 discloses a server that sets the equipment to be installed in a vehicle and used for medical checkups based on information on users who will undergo the checkups and also determines a dispatch schedule for the vehicle.

### CITATION LIST

### Patent Literature

PTL 1: JP 2021-22332 A

### SUMMARY

Medical checkups may be conducted at locations other than hospitals. Also, while a medical checkup is being conducted, transportation to a hospital may be required for a user undergoing the medical checkup.

It would be helpful to provide technology for promptly transporting a user to a hospital during a medical checkup.

An information processing apparatus according to an embodiment of the present disclosure includes a controller configured to determine, in a case in which a user whose physical condition has worsened or who is suspected of being ill is detected during a medical checkup, a hospital to which the user is to be transported from among a plurality of hospitals located within a predetermined range from a site of the medical checkup.

A system according to an embodiment of the present disclosure includes the aforementioned information processing apparatus and a terminal apparatus configured to transmit a result of the medical checkup for the user to the information processing apparatus.

A method according to an embodiment of the present disclosure includes: determining, in a case in which a user whose physical condition has worsened or who is suspected of being ill is detected during a medical checkup, a hospital to which the user is to be transported from among a plurality of hospitals located within a predetermined range from a site of the medical checkup.

According to an embodiment of the present disclosure, technology for promptly transporting a user to a hospital during a medical checkup can be provided.

### BRIEF DESCRIPTION OF THE DRAWINGS

In the accompanying drawings:

FIG. 1 is a diagram illustrating a schematic configuration of a system according to an embodiment of the present disclosure;

FIG. 2 is a block diagram of the system illustrated in FIG. 1; and

FIG. 3 is a sequence diagram illustrating operations of the system illustrated in FIG. 1.

### DETAILED DESCRIPTION

An embodiment according to the present disclosure will be described below, with reference to the drawings.

(System Configuration)

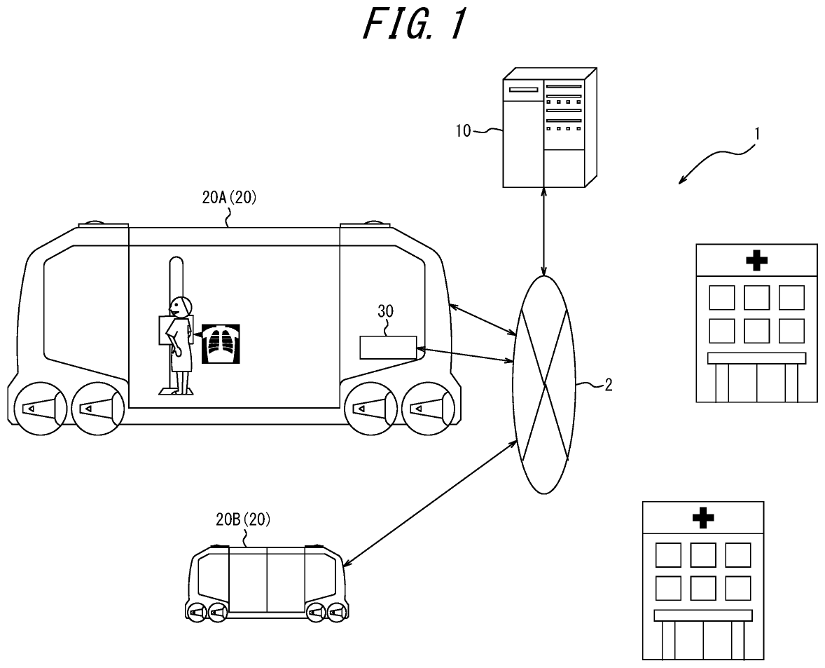

As illustrated in FIG. 1, a system 1 includes an information processing apparatus 10, vehicles 20A and 20B, and a terminal apparatus 30. In the following, the vehicles 20A and 20B also described collectively as the “vehicle 20” unless particularly distinguished.

The information processing apparatus 10, vehicle 20, and terminal apparatus 30 can communicate via a network 2. The network 2 may be any appropriate network, including a mobile communication network, the Internet, or the like.

The information processing apparatus 10 provides a medical checkup service using the vehicle 20. In a case in which a user whose physical condition has worsened or who is suspected of being ill is detected during a medical checkup, the information processing apparatus 10 determines a hospital to which the user is to be transported. The information processing apparatus 10 is, for example, a dedicated computer configured to function as a server, a general purpose personal computer, a cloud computing system, or the like.

Medical checkups can be conducted in the vehicle 20. The vehicle 20 in which medical checkups are conducted is equipped with medical equipment used in the medical checkup. Medical equipment includes, for example, blood pressure monitors, blood testing equipment, electrocardiographs, X-ray imaging equipment, ultrasound equipment, computed tomography (CT) equipment, and/or magnetic resonance imaging (MRI) equipment. The vehicle 20 patrols the area where users who receive medical checkups reside.

In the present embodiment, the medical checkup is conducted inside the vehicle 20A. The vehicle 20A is equipped with medical equipment. The vehicle 20B waits at the site of the medical checkup. In a case in which a user whose physical condition has worsened or who is suspected of being ill is detected during a medical checkup being conducted inside the vehicle 20A, the vehicle 20B transports the user to a hospital. The vehicle 20B may or may not be equipped with medical equipment.

Each vehicle 20 may be any appropriate automobile. The vehicle is, for example, a gasoline vehicle, a Battery Electric Vehicle (BEV), a Hybrid Electric Vehicle (HEV), a Plug-in Hybrid Electric Vehicle (PHEV), or a Fuel Cell Electric Vehicle (FCEV). In the present embodiment, the vehicle 20 is an Autonomous Vehicle (AV). The vehicle 20 may, however, be driven by a driver. A portion of the driving of the vehicle 20 may be automated at any level. The level of automation is, for example, one of level 1 to level 5 according to the classification of the Society of Automotive Engineers (SAE). The vehicle 20 may be a dedicated Mobility as a Service (MaaS) vehicle.

The terminal apparatus 30 is installed in the vehicle 20A in which the medical checkup is conducted. The terminal apparatus 30 is used by medical staff. The medical staff includes nurses, laboratory technicians, or the like who conduct medical checkups. In other words, the users of the terminal apparatus 30 are medical staff. The terminal apparatus 30 is, for example, a mobile phone, a smartphone, a tablet, a Personal Computer (PC), or the like.

The system 1 allows medical checkups to be conducted at locations other than hospitals by use of the vehicle 20. Here, in a case in which the medical checkup is conducted at a location other than a hospital, it is highly likely that no doctor will be present at the site of the medical checkup. In a case in which no doctor is present at the site of the medical checkup, and a user whose physical condition has worsened or who is suspected of being ill is detected during the medical checkup, the user needs to be transported to a hospital, since no doctor is present to diagnose the user. In the present embodiment, in a case in which a user whose physical condition has worsened or who is suspected of being ill is detected during a medical checkup, the information processing apparatus 10 determines a hospital to which the user is to be transported. This configuration can provide technology for promptly transporting a user to a hospital during a medical checkup.

<Information Processing Apparatus Configuration>

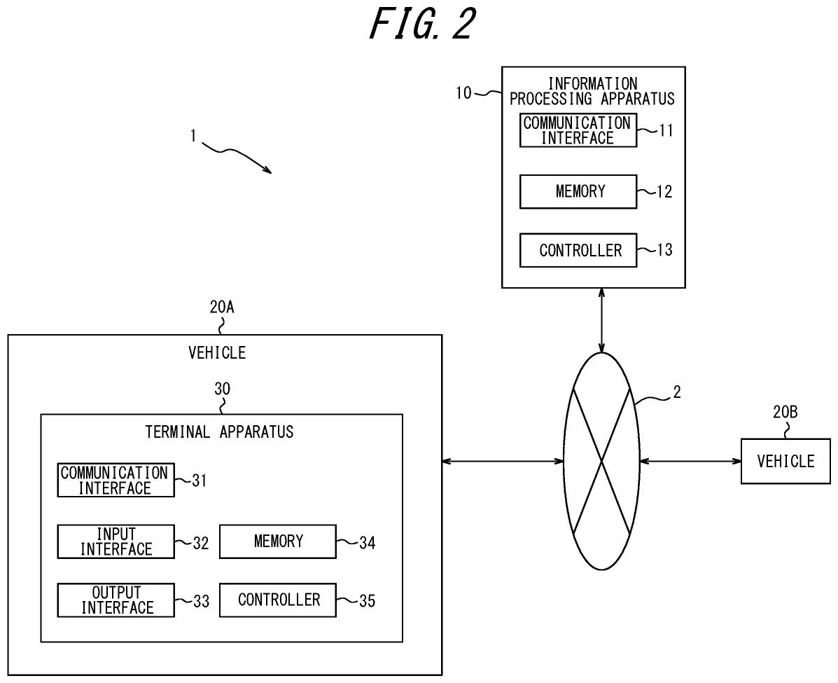

As illustrated in FIG. 2, the information processing apparatus 10 includes a communication interface 11, a memory 12, and a controller 13.

The communication interface 11 is configured to include at least one communication module for connection to the network 2. For example, the communication module is a communication module compliant with a standard such as a wired Local Area Network (LAN) or a wireless LAN. The communication module is not, however, limited to this example. Communication modules may be compliant with any appropriate communication standard. The communication interface 11 is connectable to the network 2 via the wired LAN or the wireless LAN using the communication module.

The memory 12 is configured to include at least one semiconductor memory, at least one magnetic memory, at least one optical memory, or a combination of at least two of these. The semiconductor memory is, for example, random access memory (RAM) or read only memory (ROM). The RAM is, for example, static random access memory (SRAM), dynamic random access memory (DRAM), or the like. The ROM is, for example, Electrically Erasable Programmable Read Only Memory (EEPROM) or the like. The memory 12 may function as a main memory, an auxiliary memory, a cache memory, or the like. The memory 12 stores data to be used in the operations of the information processing apparatus 10 and data obtained by the operations of the information processing apparatus 10.

The controller 13 is configured to include at least one processor, at least one dedicated circuit, or a combination thereof. Examples of the processor include a general purpose processor such as a Central Processing Unit (CPU) or a Graphics Processing Unit (GPU) and a dedicated processor dedicated to specific processing. Examples of dedicated circuits can include a Field-Programmable Gate Array (FPGA) and an Application Specific Integrated Circuit (ASIC). The controller 13 executes processes related to the operations of the information processing apparatus 10 while controlling the components of the information processing apparatus 10.

The functions of the information processing apparatus 10 may be implemented by a processor corresponding to the controller 13 executing a processing program according to the present embodiment. That is, the functions of the information processing apparatus 10 may be implemented by software. The processing program enables a computer to function as the information processing apparatus 10 by causing the computer to execute the operations of the information processing apparatus 10. That is, the computer functions as the information processing apparatus 10 by executing the operations of the information processing apparatus 10 in accordance with the processing program.

Some or all of the functions of the information processing apparatus 10 may be realized by a dedicated circuit corresponding to the controller 13. That is, some or all of the functions of the information processing apparatus 10 may be realized by hardware.

The controller 13 receives, via the network 2 from an external server, a dispatch schedule for a plurality of vehicles 20 using the communication interface 11. The controller 13 may determine a dispatch schedule for the plurality of vehicles 20 based on information on the users scheduled to receive a medical checkup. The dispatch schedule includes information such as the dispatch location and dispatch time for the vehicles 20. The dispatch location for the vehicles can be the site of the medical checkup.

Upon the start of the medical checkup, the controller 13 receives the results of the medical checkup for the user and identification information for the user from the terminal apparatus 30 via the network 2 using the communication interface 11. The results of the medical checkup for the user are, for example, X-ray images of the user. The identification information for the user may be any information that uniquely identifies the user in the system 1. The controller 13 may receive the results of the medical checkup for the user and the identification information for the user from the medical equipment installed in the vehicle 20 via the network 2 using the communication interface 11. The controller 13 stores the received result of the medical checkup for the user in the memory 12 in association with the identification information for the user.

<Terminal Apparatus Configuration>

As illustrated in FIG. 2, the terminal apparatus 30 includes a communication interface 31, an input interface 32, an output interface 33, a memory 34, and a controller 35.

The communication interface 31 is configured to include at least one communication module for connection to the network 2. The communication module is a communication module compliant with a mobile communication standard such as Long Term Evolution (LTE), 4th Generation (4G), or 5th Generation (5G). The communication module is not, however, limited to this example. Communication modules may be compliant with any appropriate communication standard.

The communication interface 31 is configured to include at least one communication module capable of communicating with the medical equipment installed in the vehicle 20. The communication module is, for example, a communication module compliant with a standard such as a wired LAN standard or a wireless LAN standard. The communication module is not, however, limited to this example. Communication modules may be compliant with any appropriate communication standard.

The input interface 32 is capable of accepting an input from a user. The input interface 32 is configured to include at least one interface for input that is capable of accepting an input from a user. The interface for input is, for example, a physical key, a capacitive key, a pointing device, a touch screen integrally provided with a display, or a microphone. However, the interface for input is not limited to these examples.

The output interface 33 is capable of outputting data. The output interface 33 is configured to include at least one interface for output that is capable of outputting data. The interface for output is, for example, a display or a speaker. The display is, for example, a Liquid Crystal Display (LCD) or an organic Electro-Luminescence (EL) display. However, the interface for output is limited to these examples.

The memory 34 is configured to include at least one semiconductor memory, at least one magnetic memory, at least one optical memory, or a combination of at least two of these. The semiconductor memory is, for example, RAM or ROM. The RAM is, for example, SRAM or DRAM. The ROM is, for example, EEPROM. The memory 34 may function as a main memory, an auxiliary memory, a cache memory, or the like. The memory 34 stores data to be used for the operations of the terminal apparatus 30 and data obtained by the operations of the terminal apparatus 30.

The controller 35 is configured to include at least one processor, at least one dedicated circuit, or a combination thereof. The processor is, for example, a general purpose processor such as a CPU or a GPU, or a dedicated processor that is dedicated to specific processing. The dedicated circuit is, for example, an FPGA or an ASIC. The controller executes processes related to operations of the terminal apparatus 30 while controlling components of the terminal apparatus 30.

The functions of the terminal apparatus 30 are realized by execution of a terminal program according to the present embodiment by a processor corresponding to the controller 35. That is, the functions of the terminal apparatus 30 are realized by software. The terminal program causes a computer to execute the operations of the terminal apparatus 30, thereby causing the computer to function as the terminal apparatus 30. That is, the computer executes the operations of the terminal apparatus 30 in accordance with the terminal program to thereby function as the terminal apparatus 30.

Some or all of the functions of the terminal apparatus 30 may be implemented by a dedicated circuit corresponding to the controller 35. That is, some or all of the functions of the terminal apparatus 30 may be realized by hardware.

The controller 35 receives the result of the medical checkup for the user and the identification information for the user from the medical equipment installed in the vehicle 20A using the communication interface 31. The controller 35 transmits the result of the medical checkup for the user and the identification information for the user to the information processing apparatus 10 via the network 2 using the communication interface 31. The controller 35 may transmit the results of the medical checkup for the user and the identification information for the user to the information processing apparatus 10 each time the controller 35 receives the results of the medical checkup for the user and the identification information for the user from the medical equipment.

(System Processes) The processes by the system 1 are described below.

<Hospital Determination Process>

In the terminal apparatus 30, the controller 35 can detect a user whose physical condition has worsened during a medical checkup. The controller 35 may detect a user whose physical condition has worsened during the medical checkup by accepting a first input and the identification information for the user from the medical staff via the input interface 32. The first input is an input indicating that a user whose physical condition has worsened is detected during the medical checkup. For example, in a case in which the physical condition of the user has worsened during blood collection, the medical staff enters the first input and the identification information for the user through the input interface 32. In a case in which a user whose physical condition has worsened is detected during the medical checkup, the controller 35 transmits a first signal and the identification information for the user to the information processing apparatus 10 via the network 2. The first signal is a signal indicating that a user whose physical condition has worsened is detected in the medical checkup.

In the terminal apparatus 30, the controller 35 can detect a user who is suspected of being ill during a medical checkup. The controller may detect a user who is suspected of being ill during the medical checkup by accepting a second input and the identification information for the user from the medical staff via the input interface 32. The second input is an input indicating that a user suspected of being ill is detected during the medical checkup. For example, in a case in which the medical staff suspects that the user is ill upon viewing an X-ray image of the user, the medical staff enters the second input and the identification information for the user through the input interface 32. The controller may detect a user suspected of being ill by analyzing the results of the medical checkup for the user received from the medical equipment. In a case in which a user who is suspected of being ill is detected during the medical checkup, the controller 35 transmits a second signal and the identification information for the user to the information processing apparatus 10 via the network 2. The second signal is a signal indicating that a user suspected of being ill is detected during the medical checkup.

In the information processing apparatus 10, the controller 13 receives the first signal or the second signal and the identification information for the user from the terminal apparatus 30 via the network 2 using the communication interface 31. In a case in which the first signal or the second signal is received, i.e., in a case in which a user whose physical condition has worsened or who is suspected of being ill is detected during a medical checkup, the controller 13 determines a hospital to which the user is to be transported.

The controller 13 determines the hospital to which the user is to be transported from among a plurality of hospitals located within a predetermined range from the site of the medical checkup. The predetermined range may be set based on the time it takes to transport the user by the vehicle 20. The controller 13 may identify the site of the medical checkup by referring to the dispatch schedule of the plurality of vehicles 20.

### Example 1

The controller 13 may determine the hospital to which the user is to be transported from among a plurality of hospitals located within a predetermined range from the site of the medical checkup based on information on the plurality of hospitals. The information on the plurality of hospitals may include any information on the plurality of hospitals. The information on the plurality of hospitals may include information on at least one affiliated hospital that is affiliated with the operator of the system 1 or the information processing apparatus 10. The controller 13 may acquire information on the plurality of hospitals by receiving information on the plurality of hospitals from an external server via the network 2 using the communication interface 11. The controller 13 may acquire information on the plurality of hospitals by receiving information on each hospital from a terminal apparatus or the like of each hospital via the network 2 using the communication interface 11. The controller 13 may acquire information on any plurality of hospitals in advance. In this case, when determining the hospital to which the user is to be transported, the controller 13 may acquire information on a plurality of hospitals located within a predetermined range from the site of the medical checkup from the information, acquired in advance, on any plurality of hospitals. Alternatively, when determining the hospital to which the user is to be transported, the controller 13 may acquire information on the plurality of hospitals by receiving the information from an external server, or from a terminal apparatus or the like at each hospital.

The information on the plurality of hospitals may include positional information on each hospital. The controller 13 may determine a hospital near the site of the medical checkup on a preferential basis as the hospital to which the user, whose physical condition has worsened or who is suspected of being ill during the medical checkup, is to be transported. This configuration enables prompt transportation of the user to the hospital. In a case in which the first signal is received, i.e., in a case in which a user whose physical condition has worsened is detected during a medical checkup, the controller 13 may determine a hospital near the site of the medical checkup on a preferential basis as the hospital to which the user is to be transported. This configuration enables a user whose physical condition has worsened to be transported promptly to a hospital, thus enabling the user to recover quickly. The controller 13 may determine the hospital nearest the site of the medical checkup as the hospital to which the user, whose physical condition has worsened or who is suspected of being ill during the medical checkup, is to be transported.

The information on the plurality of hospitals may include information on a hospital that can accept emergency patients. In a case in which the first signal is received, i.e., in a case in which a user whose physical condition has worsened is detected during a medical checkup, the controller 13 may determine the hospital that can accept emergency patients as the hospital to which the user is to be transported. This configuration enables transportation of a user whose physical condition has worsened to a hospital that can accept emergency patients. A hospital that can accept emergency patients is more likely to attend to the transported user immediately. Therefore, the user can recover quickly. In a case in which a plurality of hospitals can accept emergency patients, the controller 13 may determine a hospital near the site of the medical checkup from among the plurality of hospitals on a preferential basis as the hospital to which the user is to be transported. For example, the controller 13 determines the hospital that is closest to the site of the medical checkup from among the plurality of hospitals that can accept emergency patients as the hospital to which the user is to be transported.

### Example 2

The controller 13 may determine the hospital to which a user, whose physical condition has worsened or who is suspected of being ill during a medical checkup, is to be transported from among a plurality of hospitals located within a predetermined range from a site of the medical checkup based on the result of the medical checkup for the user and information on the plurality of hospitals. By determining the hospital based on information such as the result of the medical checkup for the user, an appropriate hospital for the health condition of the user can be determined as the hospital to which the user is to be transported.

The information on a plurality of hospitals may include information on illnesses treatable by each hospital. In this case, the controller 13 may estimate the suspected illness of the user based on the result of the medical checkup for the user. In a case in which the second signal is received, i.e., in a case in which a user suspected of being ill is detected, the controller 13 may estimate the suspected illness of the user based on the result of the medical checkup for the user. Each time the controller 13 receives the result of the medical checkup for the user from the terminal apparatus 30, the controller 13 may estimate the suspected illness of the user based on the received result of the medical checkup for the user. The controller 13 may determine the hospital that can treat the estimated, suspected illness of the user from among a plurality of hospitals located within a predetermined range from the site of the medical checkup as the hospital to which the user is to be transported. This configuration enables transportation of the user to a hospital that can treat the suspected illness of the user. In a case in which a plurality of hospitals can treat the estimated, suspected illness, the controller 13 may determine a hospital near the site of the medical checkup from among the plurality of hospitals on a preferential basis as the hospital to which the user is to be transported. For example, the controller 13 determines the hospital that is closest to the site of the medical checkup from among the plurality of hospitals that can treat the suspected illness of the user as the hospital to which the user is to be transported.

<Determination Notice Process>

In the information processing apparatus 10, upon determining the hospital to which the user is to be transported, the controller 13 generates a determination notice. The determination notice indicates that the hospital to which the user is to be transported has been determined. The determination notice may indicate the name of the hospital to which the user is to be transported. The controller 13 transmits the determination notice to the terminal apparatus 30 via the network 2 using the communication interface 11.

On the terminal apparatus 30, the controller 35 receives, via the network 2 from the information processing apparatus 10, the determination notice using the communication interface 31. The controller 35 controls the output interface 33 to output the received determination notice. For example, the controller 35 performs control for the determination notice to be displayed as an image on the display of the output interface 33 or outputted as audio from the speaker of the output interface 33. This configuration enables the medical staff to learn that the hospital to which the user is to be transported has been determined. The medical staff guides the user to the vehicle 20B or carries the user to the vehicle 20B.

<Control Signal Transmission Process>

On the information processing apparatus 10, upon determining the hospital to which the user is to be transported, the controller 13 refers to a dispatch schedule for the plurality of vehicles 20 to identify the vehicle 20B waiting at the site of the medical checkup. Upon identifying the vehicle 20B, the controller 13 generates a control signal. The control signal is a signal for causing the vehicle 20B to operate automatically and transport the user to the determined hospital. The controller 13 transmits the generated control signal to the vehicle 20B via the network 2 using the communication interface 11. The vehicle 20B receives the control signal via the network 2. The vehicle 20B transports the user to the hospital according to the control signal. Convenience can be improved by the vehicle 20B transporting the user to the hospital through automatic operation.

(System Operations)

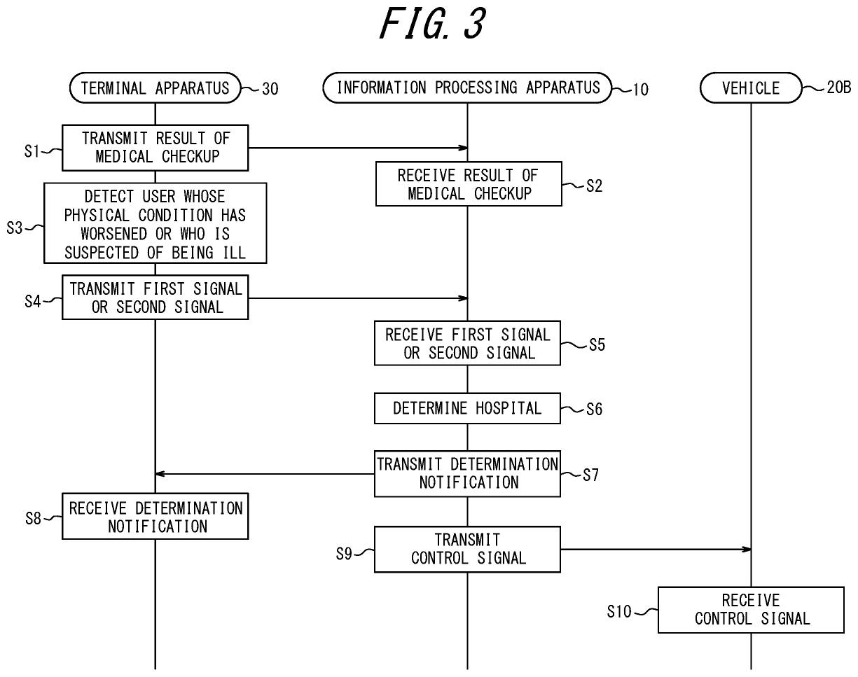

FIG. 3 is a sequence diagram illustrating operations of the system 1 illustrated in FIG. 1. These operations correspond to an example of the method according to the present embodiment. For example, when a medical checkup begins, the system 1 begins the process of step S1.

In the terminal apparatus 30, the controller 35 transmits the result of the medical checkup for the user and the identification information for the user received from the medical equipment installed in the vehicle 20A to the information processing apparatus 10 via the network 2 using the communication interface 31 (step S1). In the information processing apparatus 10, the controller 13 receives the result of the medical checkup for the user and the identification information for the user from the terminal apparatus 30 via the network 2 using the communication interface 11 (step S2).

In the terminal apparatus 30, the controller 35 detects a user whose physical condition has worsened or who is suspected of being ill during the medical checkup (step S3). The controller 35 transmits the first signal or the second signal and the identification information for the user to the information processing apparatus 10 via the network 2 using the communication interface 31 (step S4).

In the information processing apparatus 10, the controller 13 receives the first signal or the second signal and the identification information for the user from the terminal apparatus 30 via the network 2 using the communication interface 11 (step S5). The controller 13 determines the hospital to which the user is to be transported from among a plurality of hospitals located within a predetermined range from the site of the medical checkup (step S6).

On the information processing apparatus 10, the controller 13 transmits the determination notice to the terminal apparatus 30 via the network 2 using the communication interface 11 (step S7). On the terminal apparatus 30, the controller 35 receives, via the network 2 from the information processing apparatus 10, the determination notice using the communication interface 31 (step S8).

On the information processing apparatus 10, the controller 13 transmits a control signal, for controlling the vehicle 20B to drive automatically and transport the user to the determined hospital, to the vehicle 20B via the network 2 using the communication interface 11 (step S9). The vehicle 20B receives the control signal via the network 2 (step S10).

In this information processing apparatus 10, the controller 13 is configured to determine, in a case in which a user whose physical condition has worsened or who is suspected of being ill is detected during a medical checkup, a hospital to which the user is to be transported from among a plurality of hospitals located within a predetermined range from a site of the medical checkup. Here, in a case in which the vehicle 20 is used to conduct the medical checkup at a location other than a hospital, it is highly likely that no doctor will be present at the site of the medical checkup. In a case in which no doctor is present at the site of the medical checkup, and a user whose physical condition has worsened or who is suspected of being ill is detected during the medical checkup, the user needs to be transported to a hospital, since no doctor is present to diagnose the user. In the present embodiment, in a case in which a user whose physical condition has worsened or who is suspected of being ill is detected during a medical checkup, a hospital to which the user is to be transported is determined, thereby enabling prompt transportation of the user to the hospital. Thus, according to the present embodiment, technology for promptly transporting a user to a hospital during a medical checkup can be provided.

While the present disclosure has been described with reference to the drawings and examples, it should be noted that various modifications and revisions may be implemented by those skilled in the art based on the present disclosure. Accordingly, such modifications and revisions are included within the scope of the present disclosure. For example, functions or the like included in each component, each step, or the like can be rearranged without logical inconsistency, and a plurality of components, steps, or the like can be combined into one or divided.

For example, the terminal apparatus 30 may perform the processing of the information processing apparatus 10 according to the embodiment described above. In other words, in a case in which a user whose physical condition has worsened or who is suspected of being ill is detected during a medical checkup, the terminal apparatus 30 may determine a hospital to which the user is to be transported from among a plurality of hospitals located within a predetermined range from a site of the medical checkup. In this case, the terminal apparatus 30 functions as an information processing apparatus.

For example, an embodiment in which a general purpose computer functions as the information processing apparatus 10 according to the above embodiment can also be implemented. Specifically, a program in which processes for realizing the functions of the information processing apparatus 10 according to the above embodiment are written may be stored in a memory of a general purpose computer, and the program may be read and executed by a processor. Accordingly, the present disclosure can also be implemented as a program executable by a processor, or a non-transitory computer readable medium storing the program.

Examples of some embodiments of the present disclosure are described below. However, it should be noted that the embodiments of the present disclosure are not limited to these examples.

[Appendix 1] An information processing apparatus comprising a controller configured to determine, in a case in which a user whose physical condition has worsened or who is suspected of being ill is detected during a medical checkup, a hospital to which the user is to be transported from among a plurality of hospitals located within a predetermined range from a site of the medical checkup.

[Appendix 2] The information processing apparatus according to appendix 1, wherein the controller is configured to acquire information on the plurality of hospitals and determine the hospital to which the user is to be transported from among the plurality of hospitals based on the information on the plurality of hospitals.

[Appendix 3] The information processing apparatus according to appendix 1 or 2, wherein the controller is configured to acquire the information on the plurality of hospitals in advance.

[Appendix 4] The information processing apparatus according to any one of appendices 1 to 3, wherein the information on the plurality of hospitals includes positional information on each hospital among the plurality of hospitals, and the controller is configured to determine, in a case in which a user whose physical condition has worsened is detected during the medical checkup, a hospital near the site of the medical checkup on a preferential basis as the hospital to which the user is to be transported.

[Appendix 5] The information processing apparatus according to any one of appendices 1 to 4, wherein
the information on the plurality of hospitals includes information on a hospital that can accept emergency patients, and
the controller is configured to determine, in a case in which a user whose physical condition has worsened is detected during the medical checkup, the hospital that can accept emergency patients as the hospital to which the user is to be transported.

[Appendix 6] The information processing apparatus according to any one of appendices 1 to 5, wherein the controller is configured to determine the hospital to which the user is to be transported from among the plurality of hospitals based on a result of the medical checkup for the user and information on the plurality of hospitals.

[Appendix 7] The information processing apparatus according to any one of appendices 1 to 6, wherein
the information on the plurality of hospitals includes information on illnesses treatable by each hospital among the plurality of hospitals, and
the controller is configured to estimate an illness suspected for the user based on a result of the medical checkup for the user and determine a hospital that can treat the estimated illness, from among the plurality of hospitals, as the hospital to which the user is to be transported.

[Appendix 8] The information processing apparatus according to any one of appendices 1 to 7, further comprising
a communication interface, wherein
the controller is configured to transmit, via the communication interface to a vehicle waiting at the site of the medical checkup, a control signal to control the vehicle to drive automatically and transport the user to the determined hospital.

[Appendix 9] The information processing apparatus according to any one of appendices 1 to 8, wherein the controller is configured to refer to a dispatch schedule for a plurality of vehicles to identify the vehicle waiting at the site of the medical checkup.

[Appendix 10] The information processing apparatus according to any one of appendices 1 to 9, wherein the medical checkup is performed in a vehicle.

[Appendix 11] A system comprising:
the information processing apparatus according to any one of appendices 1 to 10; and
a terminal apparatus configured to transmit a result of the medical checkup for the user to the information processing apparatus.

[Appendix 12] The system according to appendix 11, wherein the terminal apparatus is configured to transmit, to the information processing apparatus, a first signal indicating detection of a user whose physical condition has worsened during the medical checkup.

[Appendix 13] The system according to appendix 11 or 12, wherein the terminal apparatus is configured to
detect a user whose physical condition has worsened during the medical checkup by accepting a first input indicating detection of a user whose physical condition has worsened during the medical checkup, and
transmit the first signal to the information processing apparatus in a case of detecting a user whose physical condition has worsened during the medical checkup.

[Appendix 14] The system according to any one of appendices 11 to 13, wherein the terminal apparatus is configured to transmit, to the information processing apparatus, a second signal indicating detection of a user who is suspected of being ill during the medical checkup.

[Appendix 15] The system according to any one of appendices 11 to 14, wherein the terminal apparatus is configured to
detect a user suspected of being ill during the medical checkup by accepting a second input indicating detection of a user suspected of being ill during the medical checkup, and
transmit the second signal to the information processing apparatus in a case of detecting a user suspected of being ill during the medical checkup.

[Appendix 16] The system according to any one of appendices 11 to 15, wherein the information processing apparatus is configured to transmit a determination notice to the terminal apparatus upon determining the hospital to which the user is to be transported.

[Appendix 17] The system according to any one of appendices 11 to 16, further comprising
a vehicle waiting at the site of the medical checkup, wherein
the information processing apparatus is configured to transmit, to the vehicle, a control signal to control the vehicle to drive automatically and transport the user to the determined hospital.

[Appendix 18] The system according to any one of appendices 11 to 17, wherein the information processing apparatus is configured to refer to a dispatch schedule for a plurality of vehicles to identify the vehicle waiting at the site of the medical checkup.

[Appendix 19] The system according to any one of appendices 11 to 18, further comprising a vehicle in which medical checkups are performed.

[Appendix 20] A method comprising: determining, in a case in which a user whose physical condition has worsened or who is suspected of being ill is detected during a medical checkup, a hospital to which the user is to be transported from among a plurality of hospitals located within a predetermined range from a site of the medical checkup.

1. An information processing apparatus comprising a controller configured to determine, in a case in which a user whose physical condition has worsened or who is suspected of being ill is detected during a medical checkup, a hospital to which the user is to be transported from among a plurality of hospitals located within a predetermined range from a site of the medical checkup.

2. The information processing apparatus according to claim 1, wherein the controller is configured to acquire information on the plurality of hospitals and determine the hospital to which the user is to be transported from among the plurality of hospitals based on the information on the plurality of hospitals.

3. The information processing apparatus according to claim 2, wherein the controller is configured to acquire the information on the plurality of hospitals in advance.

4. The information processing apparatus according to claim 2, wherein the information on the plurality of hospitals includes positional information on each hospital among the plurality of hospitals, and the controller is configured to determine, in a case in which a user whose physical condition has worsened is detected during the medical checkup, a hospital near the site of the medical checkup on a preferential basis as the hospital to which the user is to be transported.

5. The information processing apparatus according to claim 2, wherein
the information on the plurality of hospitals includes information on a hospital that can accept emergency patients, and
the controller is configured to determine, in a case in which a user whose physical condition has worsened is detected during the medical checkup, the hospital that can accept emergency patients as the hospital to which the user is to be transported.

6. The information processing apparatus according to claim 1, wherein the controller is configured to determine the hospital to which the user is to be transported from among the plurality of hospitals based on a result of the medical checkup for the user and information on the plurality of hospitals.

7. The information processing apparatus according to claim 6, wherein
the information on the plurality of hospitals includes information on illnesses treatable by each hospital among the plurality of hospitals, and
the controller is configured to estimate an illness suspected for the user based on a result of the medical checkup for the user and determine a hospital that can treat the estimated illness, from among the plurality of hospitals, as the hospital to which the user is to be transported.

8. The information processing apparatus according to claim 1, further comprising
a communication interface, wherein
the controller is configured to transmit, via the communication interface to a vehicle waiting at the site of the medical checkup, a control signal to control the vehicle to drive automatically and transport the user to the determined hospital.

9. The information processing apparatus according to claim 8, wherein the controller is configured to refer to a dispatch schedule for a plurality of vehicles to identify the vehicle waiting at the site of the medical checkup.

10. The information processing apparatus according to claim 1, wherein the medical checkup is performed in a vehicle.

11. A system comprising:
the information processing apparatus according to claim 1; and
a terminal apparatus configured to transmit a result of the medical checkup for the user to the information processing apparatus.

12. The system according to claim 11, wherein the terminal apparatus is configured to transmit, to the information processing apparatus, a first signal indicating detection of a user whose physical condition has worsened during the medical checkup.

13. The system according to claim 12, wherein the terminal apparatus is configured to
detect a user whose physical condition has worsened during the medical checkup by accepting a first input indicating detection of a user whose physical condition has worsened during the medical checkup, and
transmit the first signal to the information processing apparatus in a case of detecting a user whose physical condition has worsened during the medical checkup.

14. The system according to claim 11, wherein the terminal apparatus is configured to transmit, to the information processing apparatus, a second signal indicating detection of a user who is suspected of being ill during the medical checkup.

15. The system according to claim 14, wherein the terminal apparatus is configured to
detect a user suspected of being ill during the medical checkup by accepting a second input indicating detection of a user suspected of being ill during the medical checkup, and
transmit the second signal to the information processing apparatus in a case of detecting a user suspected of being ill during the medical checkup.

16. The system according to claim 12, wherein the information processing apparatus is configured to transmit a determination notice to the terminal apparatus upon determining the hospital to which the user is to be transported.

17. The system according to claim 11, further comprising
a vehicle waiting at the site of the medical checkup, wherein
the information processing apparatus is configured to transmit, to the vehicle, a control signal to control the vehicle to drive automatically and transport the user to the determined hospital.

18. The system according to claim 17, wherein the information processing apparatus is configured to refer to a dispatch schedule for a plurality of vehicles to identify the vehicle waiting at the site of the medical checkup.

19. The system according to claim 11, further comprising a vehicle in which medical checkups are performed.

20. A method comprising: determining, in a case in which a user whose physical condition has worsened or who is suspected of being ill is detected during a medical checkup, a hospital to which the user is to be transported from among a plurality of hospitals located within a predetermined range from a site of the medical checkup.
